(19) United States
(12) Patent Application Publication
(10) Pub. No.: US 2026/0000304 A1
(43) Pub. Date: Jan. 1, 2026

(54) METHOD AND APPARATUS FOR MONITORING BLOOD PRESSURE IN A PLURALITY OF MODES

(71) Applicants: Sukhwant Singh Khanuja, Chicago, IL (US); Deepak Pandey, Northbrook, IL (US); Steven Murakmi, Des PLaines, IL (US)

(72) Inventors: Sukhwant Singh Khanuja, Chicago, IL (US); Deepak Pandey, Northbrook, IL (US); Steven Murakmi, Des PLaines, IL (US)

(73) Assignee: CAREMATIX INC., Chicago, IL (US)

(21) Appl. No.: 18/759,950

(22) Filed: Jun. 30, 2024

(51) Int. Cl. A61B 5/021; A61B 5/00; G16H 10/60; G16H 40/67

(52) U.S. Cl. CPC A61B 5/021; A61B 5/0004; A61B 5/0022; A61B 5/742; G16H 10/60; G16H 40/67; A61B 2560/0475

(57) ABSTRACT

A method of monitoring blood pressure in a plurality of modes is provided. The method includes generating a blood pressure reading data via a blood pressure monitoring apparatus detachably attached to a user by measuring a blood pressure of the user, and time-stamping and saving the reading in a memory of the blood pressure monitoring apparatus. The method includes establishing a connection with a remote server using a cellular radio unit of the blood pressure monitoring apparatus. The method includes transmitting the blood pressure reading data to the remote server, and receiving a configuration information from the remote server, the configuration information includes of at least one or more security certificates, cloud connection details, and a setup data including activation/deactivation instruction and a multimode operation setting. The method includes operating by the blood pressure monitoring apparatus in one or more modes for monitoring the blood pressure of the user.

### BACKGROUND

The present invention generally relates to the field of medical devices. The present invention more particularly relates to method and apparatus for monitoring blood pressure in a plurality of modes.

Typically, accurate, non-invasive, continuous method of measuring blood pressure is desirable to allow individuals to monitor their blood pressure, that enables more accurate use of anti-hypertensive medication and preventing overmedication and its side effects. Even in normotensive individuals, there are those with labile hypertension usually goes undetected. Further, diseases associated with episodes of elevated blood pressure are now being detected with the use of Computerized Axial Tomography “CAT”) and Magnetic Resonance Imaging “MRI” such as multi-infarction dementia in individuals not known to be hypertensive, suggesting that monthly visits to a physician's office may not be frequent enough to ensure that an individual is not periodically hypertensive. It is becoming evident that the need for more frequent blood pressure monitoring in the so-called normotensive population is greater than heretofore recognized. Additionally, having a record of a patient's blood pressure at various points during the day may help in diagnosis and treatment of patients suffering from hypotension as well as from hypertension, and from disorders related to non-normal blood pressure behavior. Also, hypotension most often results in fainting, syncope, or general feelings of weakness or lethargy, and can lead to potentially dangerous losses of functioning. Wherever the patterns of increasing or decreasing blood pressure can be established and treated, they can be controlled before they can cause damage to the individual.

The above-mentioned shortcomings, disadvantages and problems are addressed herein, and will be understood by reading and studying the following specification.

### SUMMARY

This summary is provided to introduce a selection of concepts in a simplified form that are further disclosed in the detailed description. This summary is not intended to determine the scope of the claimed subject matter.

The embodiments herein address the above-recited needs for a method and an apparatus for monitoring blood pressure in a plurality of modes.

In one aspect a method of monitoring blood pressure in a plurality of modes is provided. The method includes generating a blood pressure reading via a blood pressure monitoring apparatus detachably attached to a user by measuring a blood pressure of the user, and time-stamping and saving the reading in a memory of the blood pressure monitoring apparatus. The method also includes establishing a connection with a remote server using a cellular radio unit of the blood pressure monitoring apparatus. The method also includes transmitting the blood pressure reading to the remote server, and receiving a configuration information from the remote server, the configuration information includes of at least one or more security certificates, cloud connection details, and a setup data including activation/deactivation instruction and a multimode operation setting. The method also includes operating by the blood pressure monitoring apparatus in one or more modes for monitoring the blood pressure of the user based on the configuration information.

In an embodiment, manually select the multimode operation setting by the user, where the multimode operation setting includes configuration of at least one of a fixed interval or variable intervals between a plurality of readings, with a pre-configured relaxation time in between the readings.

In an embodiment, the one or more modes includes a first mode where the intervals between the reading ranges between 0 to 4000 seconds, where one of same or different intervals is selected between a plurality of readings.

In an embodiment, the one or more modes includes a second mode where the intervals between the reading ranges about 0 to 4000 seconds and about 1 to 99 readings are taken with an interval between a plurality of readings.

In an embodiment, using the configuration information to determine if blood pressure monitoring device needs to ping the remote server for one or more additional configuration parameters including at least a new security certificate to encrypt the communication with the remote server, new server configuration and a new firmware for the cellular radio unit.

In an embodiment, performing one of activating or deactivating a display of a user interface of the blood pressure monitoring device based on the setup data, where a message is displayed to the user via the user interface, An example of the message is, the reading is taken but not displayed.

In an embodiment, displaying at least one of a countdown of time between the readings and a current reading and automatically taking a subsequent reading, and the display has a countdown of time between a current reading and a subsequent reading.

In another aspect a blood pressure monitoring apparatus is provided. The apparatus includes a blood pressure subsystem configured for generating a blood pressure reading via a blood pressure detection unit detachably attached to a user by measuring a blood pressure of the user, and time-stamping and saving the reading in a memory. The apparatus also includes a microcontroller unit for establishing a connection with a remote server via a cellular radio unit. The apparatus also includes a cellular radio unit for transmitting the blood pressure reading to the remote server, and receiving a configuration information from the remote server, where the configuration information includes of at least one or more security certificates, cloud connection details, and a setup data including activation/deactivation instruction and a multimode operation setting and the blood pressure monitoring apparatus operates in one or more modes for monitoring the blood pressure of the user based on the configuration information.

In an embodiment, the memory is a non-volatile memory.

In an embodiment, the apparatus includes a mode selection button to enables a user to manually select the multimode operation setting, where the multimode operation setting includes configuration of at least one of a fixed interval or variable intervals between a plurality of readings, with a pre-configured relaxation time in between the readings.

In an embodiment, the one or more modes includes a first mode where the intervals between the reading ranges between 0 to 4000 seconds, where one of same or different intervals is selected between a plurality of readings.

In an embodiment, the one or more modes includes a second mode where the intervals between the reading ranges between 0 to 4000 seconds and 1 to 99 readings are taken with an interval between a plurality of readings.

In an embodiment, the configuration information determines if blood pressure monitoring device needs to ping the remote server for one or more additional configuration parameters including at least a new security certificate to encrypt the communication with the remote server, new server configuration and a new firmware for the cellular radio unit.

In an embodiment, the apparatus further configured to perform one of activating or deactivating a display of a user interface based on the setup data, where a message is displayed to the user via the user interface indicating that the reading is taken but not displayed.

In an embodiment, the apparatus further configured to display at least one of a countdown of time between the readings and a current reading and automatically taking a subsequent reading.

The various embodiments of the present technology provide method and apparatus for measuring blood pressure in multiple modes, i.e., automatically take multiple blood pressure readings in a consecutive manner, with a pre-configured relaxation time between successive blood pressure readings. The present disclosure envisages a blood pressure monitor comprising a microcontroller unit (MCU), power supply, BP subsystem, such as pumps and sensors, display, memory, and a cellular radio module. The microcontroller unit communicates with the cellular radio module. The blood pressure monitor takes blood pressure measurements using an arm cuff, creates a corresponding timestamp, and stores the reading until a connection is established with a secured cloud environment through the cellular radio module. The blood pressure monitor supports a plurality of users. In accordance with the present disclosure, a user could manually enable the multimode operation of the blood pressure monitor and configure the number of readings to be taken and the interval durations. The user could also configure the interval (in seconds) between consecutive blood pressure readings. The intervals can be configured for the range 0-255 seconds.

It is to be understood that the aspects and embodiments of the disclosure described above may be used in any combination with each other. Several of the aspects and embodiments may be combined to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

These and other objects and advantages will become more apparent when reference is made to the following description and accompanying drawings.

### BRIEF DESCRIPTION OF THE DRAWINGS

The other objects, features and advantages will occur to those skilled in the art from the following description of the preferred embodiment and the accompanying drawings in which:

FIG. 1 depicts a block diagram of an example environment suitable for practicing the systems and methods described herein;

FIG. 2 depicts a block diagram representation of the blood pressure monitoring apparatus, in accordance with an embodiment;

FIGS. 3A-3B depicts a flow diagram depicting a process of configuring the blood pressure monitoring apparatus remotely from a cloud server, in accordance with an embodiment;

FIGS. 4A-4C depict user interface views of operating the blood pressure monitoring apparatus in various modes, in accordance with an exemplary scenario; and

FIG. 5 illustrates a flow diagram depicting a method of monitoring blood pressure of a user in a plurality of modes, in accordance with an embodiment.

Although the specific features of the embodiments herein are shown in some drawings and not in others. This is done for convenience only as each feature may be combined with any or all of the other features in accordance with the embodiments herein.

### DETAILED DESCRIPTION

The detailed description of various exemplary embodiments of the disclosure is described herein with reference to the accompanying drawings. It should be noted that the embodiments are described herein in such details as to clearly communicate the disclosure. However, the amount of details provided herein is not intended to limit the anticipated variations of embodiments; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail below. It should be understood, however, that it is not intended to limit the disclosure to the forms disclosed, but on the contrary, the disclosure is to cover all modifications, equivalents, and alternatives falling within the scope of the disclosure.

The detailed description of various exemplary embodiments of the disclosure is described herein with reference to the accompanying drawings. It should be noted that the embodiments are described herein in such details as to clearly communicate the disclosure. However, the details provided herein is not intended to limit the anticipated variations of embodiments; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail below. It should be understood however, it is not intended to limit the disclosure to the forms disclosed, but on the contrary, the disclosure is to cover all modifications, equivalents, and alternatives falling within the scope of the disclosure.

The various embodiments of the present technology provide method and apparatus for measuring blood pressure in multiple modes, i.e., automatically take multiple blood pressure readings in a consecutive manner, with a pre-configured relaxation time between successive blood pressure readings. The present disclosure envisages a blood pressure monitor comprising a microcontroller unit (MCU), power supply, BP subsystem, such as pumps and sensors, display, memory, and a cellular radio module. The microcontroller unit communicates with the cellular radio module. The blood pressure monitor takes blood pressure measurements using an arm cuff, creates a corresponding timestamp, and stores the reading until a connection is established with a secured cloud environment through the cellular radio module. The blood pressure monitor supports a plurality of users. In accordance with the present disclosure, a user could manually enable the multimode operation of the blood pressure monitor and configure the number of readings to be taken and the interval durations. The user could also configure the interval (in seconds) between consecutive blood pressure readings. The intervals can be configured for the range 0-255 seconds.

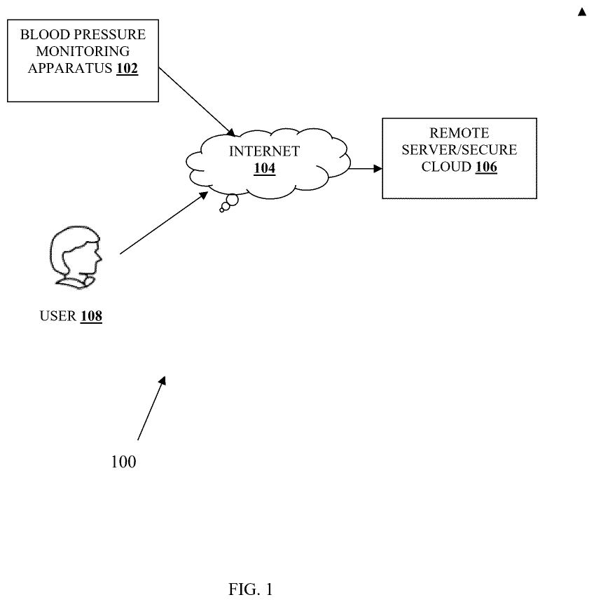

FIG. 1 depicts a block diagram of an example environment 100 suitable for practicing the systems and methods described herein. It should be noted, however, that the environment 100 is just an example and is simplified embodiment provided for illustrative purposes and reasonable deviations of this embodiment are possible as will be evident to those skilled in the art. As shown in FIG. 1, the environment may include a blood pressure monitoring apparatus 102 communicatively coupled via a network/internet 104 to a remote server/secure cloud 106. The network 104 includes, for example, a cellular radio network. One of more blood pressure detecting units associated with the blood pressure monitoring apparatus 102 may be detachably attached to the body of a user, for example, a wrist of the user. A user 108 may be able to access the remote server 106 via the internet 104 using a client device. In some embodiments, the client device may include but is not limited to, a laptop computer, a tablet computer, a desktop computer, a mobile phone, and so forth. In some embodiments, the remote server 106 may be a standalone computing device. In some other embodiments, the remote server 106 may be implemented as a cloud-based computing resource shared by multiple users. The cloud-based computing resource(s) can include hardware and software available at a remote location and accessible over a network (for example, the Internet). The cloud-based computing resource(s) can be dynamically re-allocated based on demand. The cloud-based computing resources may include one or more server farms/clusters including a collection of computer servers which can be co-located with network switches and/or routers.

In some embodiments, the blood pressure reading is time stamped and saved in a memory associated with the blood pressure monitoring apparatus 102. The blood pressure monitoring apparatus 102 includes a cellular radio unit configured for establishing a connection with the remote server 106. After the connection is established, the blood pressure monitoring apparatus 102 transmits the blood pressure reading to the remote server 106 and receives a configuration information from the remote server 106. The configuration information includes of at least one or more security certificates, cloud connection details, and a setup data comprising activation/deactivation instruction and a multimode operation setting. The blood pressure monitoring apparatus 102 operates in one or more modes for monitoring the blood pressure of the user based on the configuration information.

In some embodiments, the blood pressure monitoring apparatus 102 is remotely configured vie the remote server 106 for at least one of: an activation/deactivation status, automatic multiple reading operation, device topics, Internet of things (IOT) hub/device security certificates, or a cellular radio firmware update. In some embodiments, when the blood pressure monitoring apparatus 102 is powered ON, the blood pressure monitoring apparatus 102 pins the remote server 106 via the network cloud and a response received from the remote server 106 determines if an action needs to be taken to receive any additional configuration data. After pinging the remote server, the blood pressure monitoring apparatus 102 connects to an IOT Hub and reads a device twin to the setup data message. In some other embodiments, when the blood pressure monitoring apparatus 102 transmits reading records to the IOT hub, the blood pressure monitoring apparatus 102 reads the twin data from the Hub. The twin data provides the blood pressure monitoring apparatus 102 with the setup data message and a configuration value. The blood pressure monitoring apparatus 102 uses the setup data message to change its activation status or to get information needed to set up for multimode operation. The blood pressure monitoring apparatus 102 uses the configuration value to determine if it needs to ping the remote server 106 for additional configuration parameters. The additional parameters include but is not limited to new security certificates to encrypt the communication with the IoT Hub, new IoT Hub configuration (device topics for use when publishing or subscribing), new firmware for the cellular radio. This is the same information the blood pressure monitoring apparatus 102 might receive when pinging the remote server/cloud 106 during a power cycle.

In some embodiments, the blood pressure monitoring apparatus 102 pings the remote server/cloud 106 and receives a ping response that can be used to control the blood pressure monitoring apparatus 102 to take more action to receive: new IoT Hub topics, new security certificates or new cellular radio firmware updates. In some embodiments, the IoT Hub keeps a copy of what is called the twin data for each device/blood pressure monitoring apparatus 102 registered with it. The twin data is used so the blood pressure monitoring apparatus 102 can see what was “reported configuration” by the blood pressure monitoring apparatus 102 and what is “desired configuration” by the IoT Hub. When the blood pressure monitoring apparatus 102 reads its twin from the IoT Hub, it will receive the setup data message and configuration flag as the “desired configuration” changes. The blood pressure monitoring apparatus 102 uses the setup data to determine if it should be in an active or deactivated state and to see if it should enter multimode operation or normal operation. The blood pressure monitoring apparatus 102 uses the configuration flag to determine if it needs to ping the remote server 106 to receive any additional configuration.

In some embodiments, the setup data received from the remote server 106 informs the blood pressure monitoring apparatus 102 of its activation status. When activated the blood pressure monitoring apparatus 102 performs normal operations. When deactivated the blood pressure monitoring apparatus 102 will still take blood pressure readings but will not display the reading to the user on the blood pressure monitoring apparatus 102 LCD screen (for example, an error E7 is displayed). The reading record will be transmitted to the IoT Hub.

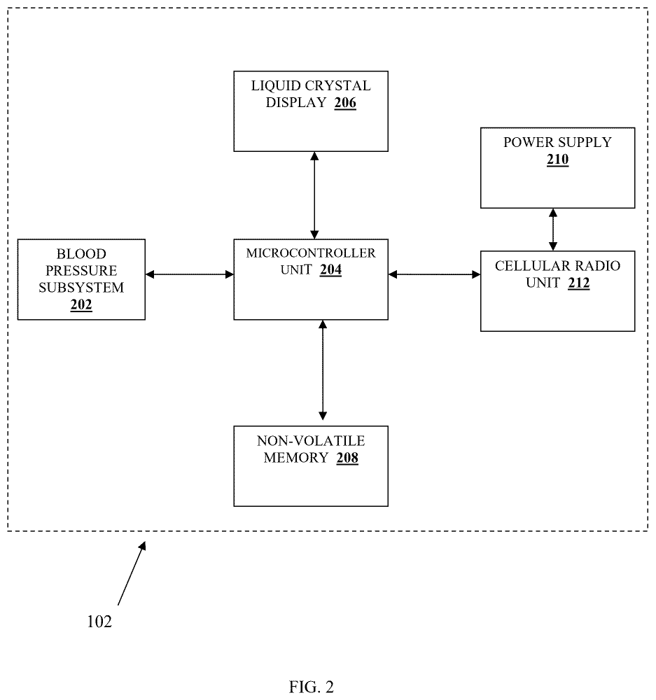

FIG. 2 depicts a block diagram representation of the blood pressure monitoring apparatus, in accordance with an embodiment. The blood pressure monitoring apparatus 102 includes a blood pressure subsystem 202, a microcontroller unit 204, a liquid crystal display 206/user interface, a non-volatile memory 208, a power supply 210 and a cellular radio unit 212. The blood pressure subsystem 202 is configured for generating a blood pressure reading via a blood pressure detection unit detachably attached to a user 108 by measuring a blood pressure of the user, and time-stamping and saving the reading in a memory. The microcontroller unit 204 is configured for establishing a connection with a remote server 106 via, for example, the cellular radio unit 212. The power supply 210 is used to provide power to the blood pressure monitoring apparatus 102. The cellular radio unit may be used to 212 transmit the blood pressure reading to the remote server 106 by, for example, a cellular radio network. After receiving configuration information from the remote server 106. The configuration information includes, but is not limited to, at least one of one or more security certificates, cloud connection details, and a setup data comprising activation/deactivation instruction and a multimode operation setting. The configuration information is evaluated and used to determine if the blood pressure monitoring device needs to request the remote server for one or more additional configuration parameters and generate an at least a new security certificate. To encrypt the communication with the remote server, new server configuration, and a new firmware for the cellular radio unit, the blood pressure monitoring apparatus 102 operates in one or more modes for monitoring the blood pressure of the user 108 based on the configuration information. The memory is a non-volatile memory 208.

In some embodiments, the blood pressure monitoring apparatus 102 includes a mode selection button which enables the user 108 to manually select the at least one multimode operation setting. The multimode operation setting comprises configuration of at least one of a fixed interval or variable intervals between a plurality of readings, with a pre-configured relaxation time in between the readings. In some embodiments the configuration information may include a configuration message communicated as a part of the apparatus 102 to server communication protocol, such that the protocol is implemented in apparatus 102. The workflow to check and update the configuration changes remotely is in-built in the apparatus 102. In some embodiments, the configuration message may be fixed based or adaptable. For example, for adaptable, if a user takes a reading which is high, the next time they may be asked to take 3 readings 1 minute apart to see how the average reading is as compared to normal. If the average is normal, the BP unit may receive a message to go back to taking just one reading very time. This logic would be in the server.

For fixed configuration, if a physician suggests a user to take 28 readings 30 minutes apart over 14 hours to see how the user's blood pressure readings change over time during the day, this could be a fixed configuration message. Another use case is when a user needs to measure orthostatic drop in blood pressure when they stand up. The first reading is taken while sitting and the blood pressure unit takes a 2nd reading 45 sec (or 60 sec) apart where the user has been instructed to stand-up during the period.

In some embodiments, the one or more modes includes a first mode where the intervals between the reading ranges between 0 to 4000 seconds and one of same or different intervals is selected between a plurality of readings and a second mode where the intervals between the reading ranges between 0 to 4000 seconds and 1 to 99 readings are taken with an interval between a plurality of readings.

In some embodiments, a multimode operation is used to configure the blood pressure monitoring apparatus 102 to automatically take several blood pressure readings in a row with a configured relaxation time in between the readings. Every time the blood pressure monitoring apparatus 102 communicates with the remote server 106, it receives an update of the setup data including information for multimode operation. Subsequently, the user 108 or a server logic can enable multimode remotely by logging into a portal application and configuring the number of readings and the interval durations. In some embodiments, the multimode operation can be configured for multiple users. The multimode cannot be configured for a guest user.

The blood pressure monitoring apparatus 102 includes one or more user buttons. In an example embodiment, the blood pressure monitoring apparatus 102 has two user buttons, such that when one is pressed, it turns on and takes a reading. If the configuration file as sent by the remote server 106 to the blood pressure monitoring apparatus 102 has multimode operation turned on, the blood pressure monitoring apparatus 102 will take defined number of X readings, each reading being Y seconds apart. X and Y are specified in configuration file. Each reading is transmitted with the device serial number and user flag. This is received at the remote server 106 and matched to the user ID. The blood pressure monitoring apparatus 102 also includes a guest button that allows the user to login as a guest. The blood pressure monitoring apparatus 102 allows the user to login as guest and use multimode operation but does not transmit the guest readings to the remote server 106. This way it does not get recorded in the user's account by mistake. The remote server 106 sends a message to the blood pressure monitoring apparatus 102 activating the multimode. The message contains the number of readings (X) and time interval (Y) between each reading. When the blood pressure monitoring apparatus 102 talks to the remote server 106, it receives this message. When the user takes a reading, the blood pressure monitoring apparatus 102 will take the X number of readings. After taking each reading it will display the Y time and countdown to 0 and automatically take the next reading till X number of readings are taken.

In an exemplary scenario, the blood pressure monitoring apparatus 102 takes three readings and the interval between each reading can change for example, Reading 1, wait 60 seconds, Reading 2, wait 45 seconds, Reading 3. In another exemplary scenario, the interval between each reading is fixed at Y seconds. X can be any number below 99. All readings stored in the remote server/cloud 106 are accessible by permission and protected by login/password authentication. In some embodiments the readings can be accessed by a physician, caregiver or family member by logging into the remote server 106.

In some embodiments, the blood pressure monitoring apparatus 102/the remote server 106 is configured to perform one of activating or deactivating a display of a user interface based on the setup data, where a message is displayed to the user via the user interface indicating that the reading is taken but not displayed. In an embodiment, the blood pressure monitoring apparatus 102 is configured to display at least one of a countdown of time between the readings and a current reading and automatically taking a subsequent reading. Based on the multimode setting, the display (used interchangeably with the term “LCD” or user interface) depicts the countdown of time between readings and then automatically to take the next reading. The display shows the reading number corresponding to a current reading, for example 3 of 24. The readings may be labelled if needed. For example, if the user is required to take two readings 45 seconds apart where the first is tilling and the second one is standing, the display shows 1 of 2 and shows “sitting” and takes the first reading, then displays countdown from 45 seconds and shows “stand up”, then shows 2 of 2 and shows “standing and takes the second reading. An example of the setup data is shown below:

### Setup Data for Multimode Operation (Type 1):

Data Value

Last Update
Timestamp
1529897345
Active Flag
Activate
User1 BP Mode
3
User1 Interval-12
60
User1 Interval-23
120
User2 BP Mode
3
User2 Interval-12
60
User2 Interval-23
120

An example setup data for multimode operation (type 2) is shown below:

Data Value

Last Update
Timestamp
1529897345
Active Flag
Activate
User1
(number of readings)
6
User1 Interval
(seconds)
120
User2
(number of readings)
5
User2 Interval
(seconds)
120

An example of ping service response data is shown below:

Description

Status. Possible values
0 = No new config items available.
1 = New config item(s) available to
download
Indicates new Root CA certificate is
available to download. Value contains the
size of the certificate
Indicates new Client certificate is available
to download. Value contains the size of the
certificate
Indicates new Client Key is available to
download. Value contains the size of the key
Indicates new IoT Hub Host is available to
download. Value contains the size of the
JSON data
Indicates new cellular radio module
firmware version to download.
Microcontroller firmware version number
used by the device.
Protocol version number used by the device.
Version number of the new IoT Hub
Configuration
Token String to be used to call Cloud
services to get information about the
changes.

An example of a twin response data is shown below:

| Object | Description |
|---|---|
| Desired | Setup data. It can be empty string if there is no |
| | new setup data to communicate to the device. |
| | See section 3.1 for Setup Data format. |
| | Configuration Version of the device at the |
| | server side. This is version number of the |
| | configuration data and certificates used to |
| | communicate with the Cloud Server. |
| | This is system-maintained version number. |
| | Application can't update it. |
| Reported | Microcontroller firmware version number used |
| | by the device. |
| | Protocol version number used by the device. |
| | Configuration version using by the device to |
| | communicate with the IoT Hub |
| | Firmware version of Cellular module |
| | SIM Card Number |
| | Module's IMEI Number |
| | SIM provider's IMSI number |
| | This is system-maintained version number. |
| | Application can't update it. |

In some embodiments, the remote server 106 updates the “Desired” section whereas the blood pressure monitoring apparatus 102 maintains “Reported” section only. In some embodiments, the remote server 10t6 sends a compliance reminder to the user via test message or phone calls in case the user is not close to the blood pressure monitoring apparatus 102 and does not see the displayed alert signal. The user still has to press the start button on the device multiple times for multiple readings. In some embodiments, when the user presses the start button, the apparatus 102 automatically takes a number of readings in configuration file (say for example, 2 or 3 or 24 for example) in intervals as defined in configuration file (say for example, 45 sec, 60 sec etc. apart). All alert and compliance reminders are sent vis text or phone calls or an app in case the user downloads it. In the present apparatus, the configuration file can also have other parameters like turning off the display so the readings are not displayed on the screen but only transmitted to server. Further, the configuration setting to take readings periodically are specified by user. This is usually done by pressing buttons or a switch on the device which can be operated by the user. In the present technology, the configuration setting is sent by remote server to the user device. The user device will then operate as specified in the configuration setting (or parameter file) downloaded. The configuration file is pushed from the remote server 106 to the blood pressure monitoring apparatus 102. Once the user presses the start button the blood pressure monitoring apparatus 102 operates as specified in configuration file (e.g., turn off display, take multiple readings, etc). All this is done automatically without further action by user.

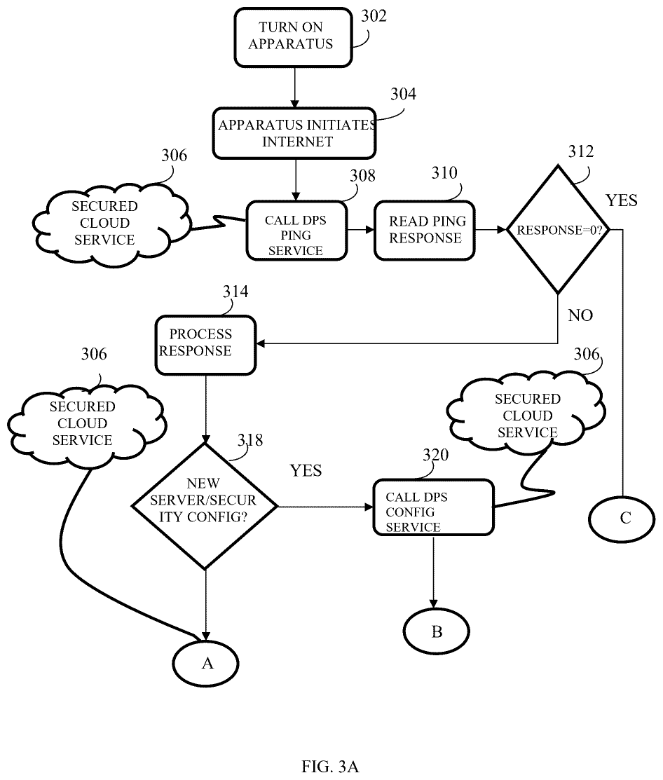

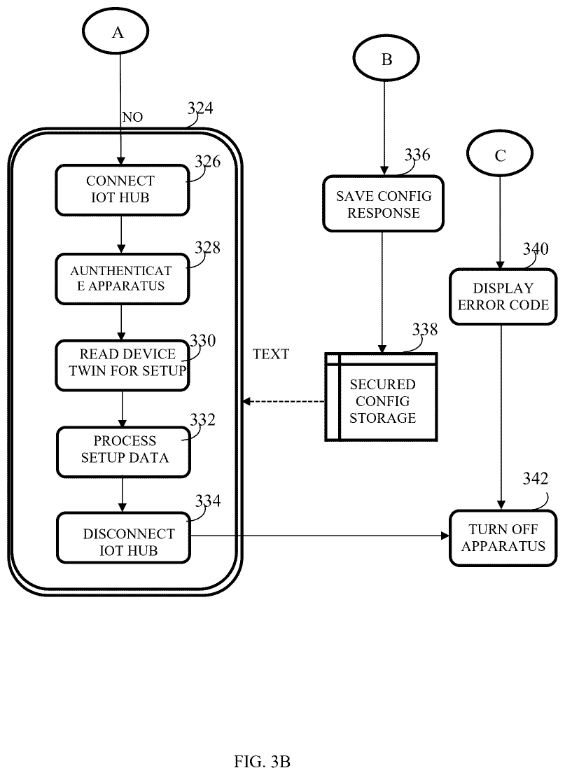

FIGS. 3A-3B depicts a flow diagram depicting a process of configuring the blood pressure monitoring apparatus remotely from a cloud server, in accordance with an embodiment. At step 302, the blood pressure monitoring apparatus 102 is turned ON. At step 304, the blood pressure monitoring apparatus 102 initiates internet. At step 306, the blood pressure monitoring apparatus 102 pings the secured cloud service (remote server 106) by calling a DPS ping service. At step 310, a ping response is read by the blood pressure monitoring apparatus 102. At step 312 it is checked if response is null. If the response is not null, the at step 314 the response is processed. At step 318, it is checked if there is a new server or security configuration in the response. If there is a new server or security configuration in the response, then at step 320 DPS config service is called. If at step 312, the response is null, then at step 340 an error code is displayed at the LCD 206 of the blood pressure monitoring apparatus 102. Is at step 318, there is no new server or security configuration, then the control transfers to the IOT hub 324 is performed. At step 326, the blood pressure monitoring apparatus 102 connects to an IOT hub. At step 328, the blood pressure monitoring apparatus 102 is authenticated. At step 330, the device twin is read for setup. At step 332, the set up data is processed. At step 334 the blood pressure monitoring apparatus 102 disconnects from the IOT hub. At step 336, the configuration response is saved. At step 338 the secured configuration is stored as text in the IOT hub 324.

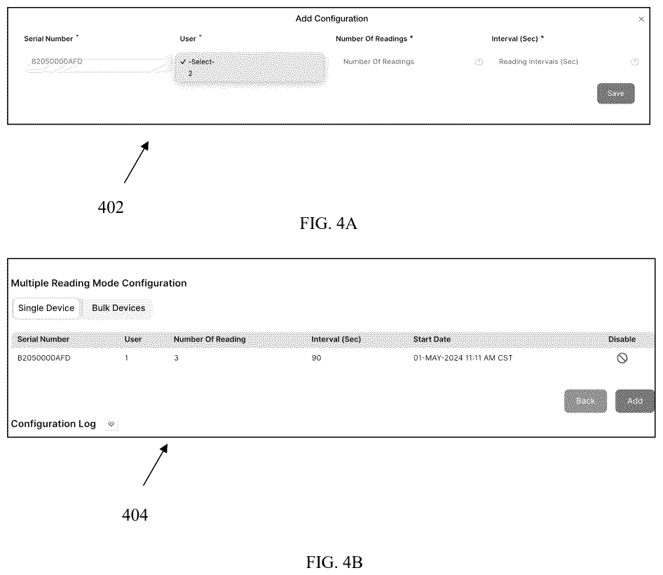

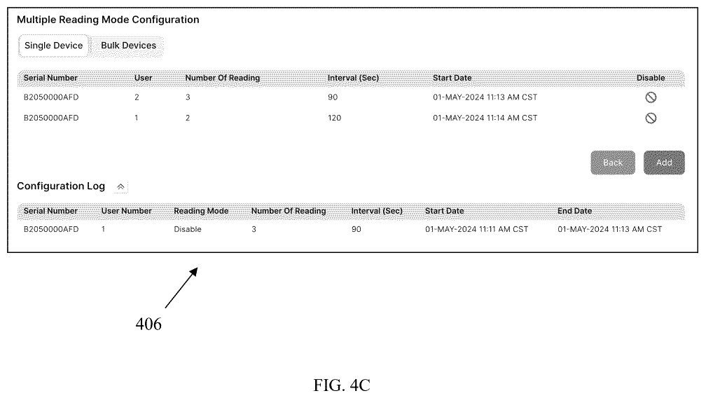

FIGS. 4A-4C depicts user interface views of operating the blood pressure monitoring apparatus in various modes, in accordance with an exemplary scenario. As depicted in the exemplary user interface view 402 of FIG. 4A, when the user 108 is allowed to select a number of readings from the drop down menu and save. As shown in the user interface view 404 of FIG. 4B, the user1 108 selects 3 number of readings at an interval of 90 seconds. FIG. 4C shows a user interface view 406 of multiple reading mode configuration. As shown in FIG. 4C, the users 1 and 2 select 3 and 2 number of readings at an interval of 90 and 120 seconds respectively, via single device with a common device serial number.

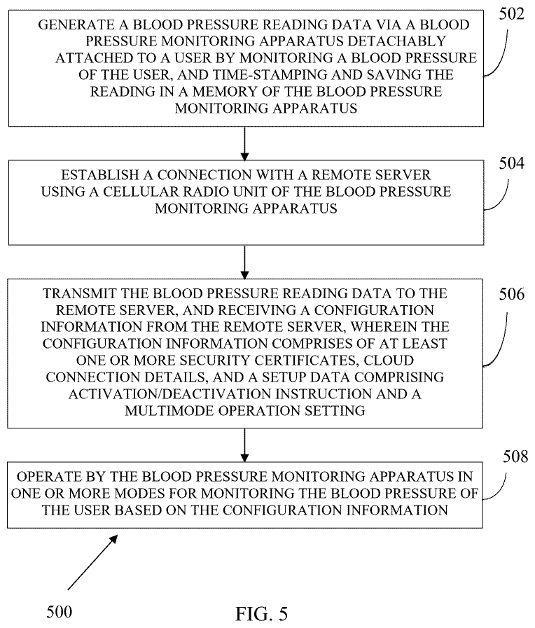

FIG. 5 illustrates a flow diagram depicting a method of monitoring blood pressure of a user in a plurality of modes, in accordance with an embodiment. At step 502, a blood pressure reading is generated via a blood pressure monitoring apparatus detachably attached to a user by measuring a blood pressure of the user, and time-stamping and saving the reading in a memory of the blood pressure monitoring apparatus. At step 504, a connection is established with a remote server using a cellular radio unit of the blood pressure monitoring apparatus. At step 506, the blood pressure reading is transmitted to the remote server, and a configuration information is received from the remote server, where the configuration information includes of at least one or more security certificates, cloud connection details, and a setup data including activation/deactivation instruction and a multimode operation setting. At step 508, operated by the blood pressure monitoring apparatus in one or more modes for monitoring the blood pressure of the user based on the configuration information.

As used herein, “about” or “approximately” shall generally mean within 20 percent, preferably within 10 percent, and more preferably within 5 percent of a given value or range. Other than in the operating examples, or where otherwise indicated, all numbers expressing quantities of ingredients and/or reaction conditions are to be understood as being modified in all instances by the term “about”.

The embodiments herein can include both hardware and software elements. The embodiments that are implemented in software include but are not limited to, firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The system, method, computer program product, and propagated signal described in this application may, of course, be embodied in hardware; e.g., within or coupled to a Central Processing Unit (“CPU”), microprocessor, microcontroller, System on Chip (“SOC”), or any other programmable device. Additionally, the system, method, computer program product, and propagated signal may be embodied in software (e.g., computer readable code, program code, instructions and/or data disposed in any form, such as source, object or machine language) disposed, for example, in a computer usable (e.g., readable) medium configured to store the software. Such software enables the function, fabrication, modeling, simulation, description and/or testing of the apparatus and processes described herein.

Such software can be disposed in any known computer usable medium including semiconductor, magnetic disk, optical disc (e.g., CD-ROM, DVD-ROM, and the like) and as a computer data signal embodied in a computer usable (e.g., readable) transmission medium (e.g., carrier wave or any other medium including digital, optical, or analog-based medium). As such, the software can be transmitted over communication networks including the Internet and intranets. A system, method, computer program product, and propagated signal embodied in software may be included in a semiconductor intellectual property core (e.g., embodied in HDL) and transformed to hardware in the production of integrated circuits. Additionally, a system, method, computer program product, and propagated signal as described herein may be embodied as a combination of hardware and software.

A “computer-readable medium” for purposes of embodiments of the present invention may be any medium that can contain, store, communicate, propagate, or transport the program for use by or in connection with the instruction execution system, apparatus, system or device. The computer readable medium can be, by way of example only but not by limitation, an electronic, magnetic, optical, electromagnetic, infrared, or semiconductor system, apparatus, system, device, propagation medium, or computer memory.

A “processor” or “process” includes any human, hardware and/or software system, mechanism or component that processes data, signals or other information. A processor can include a system with a general-purpose central processing unit, multiple processing units, dedicated circuitry for achieving functionality, or other systems. Processing need not be limited to a geographic location or have temporal limitations. For example, a processor can perform its functions in “real time,” “offline,” in a “batch mode,” etc. Portions of processing can be performed at different times and at different locations, by different (or the same) processing systems.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims. The scope of the embodiments will be ascertained by the claims to be submitted at the time of filing a complete specification.

What is claimed is:

1. A method of measuring blood pressure in a plurality of modes, the method comprising:
a) generating a blood pressure reading via a blood pressure monitoring apparatus;
time-stamping and saving the reading in a memory of the blood pressure monitoring apparatus;
b) establishing a connection with a remote server using a cellular radio unit of the blood pressure monitoring apparatus;
c) transmitting the blood pressure reading data to the remote server;
d) receiving an at least one configuration information from the remote server;
e) evaluate the configuration information; and
f) determine if the blood pressure monitoring apparatus needs to request the remote server for one or more additional configuration parameters and generate an at least one new security certificate.

2. The method of claim 1, wherein the blood pressure monitoring apparatus comprises a mode selection button for at least one multimode operation setting.

3. The method of claim 2, wherein the multimode operation setting comprises one or more modes, wherein a first mode has intervals between the reading ranges between 0 to 4000 seconds.

4. The method of claim 2, wherein the multimode operation setting comprises one or more modes, wherein a second mode has intervals between the reading ranges between about 0 to 4000 seconds and about 1 to 99 readings taken with an interval between a plurality of readings.

5. The method of claim 1, wherein the at least a new security certificate encrypts the communication with the remote server, new server configuration and a new firmware for the cellular radio unit.

6. The method of claim 1, wherein the blood pressure monitoring apparatus includes a display of a user interface, the display of the user interface can be activated or deactivated based on the setup data, and a message is displayed to the user via the user interface.

7. The method of claim 6, wherein the display has a countdown of time between a current reading and a subsequent reading.

8. A blood pressure monitoring apparatus comprising:
a) a blood pressure subsystem for generating a blood pressure reading;
b) a blood pressure detection unit detachably attached to a user[
c) a time-stamp;
d) a memory;
e) a microcontroller unit;
f) a remote server; and
g) a cellular radio unit.

9. The apparatus of claim 8, wherein the apparatus comprises a mode selection button which enables a user to manually select the multimode operation setting.

10. The apparatus of claim 9, wherein the multimode operation setting comprises configuration of at least one of a fixed interval or a variable interval(s) between a plurality of readings, with a pre-configured relaxation time between the readings.

11. The apparatus of claim 9, wherein the one or more modes comprises a first mode having intervals between the reading ranges of about 0 to 4000 seconds, and one of a same or a different interval is selected between a plurality of readings.

12. The apparatus of claim 9, wherein the one or more modes comprises a second mode having the intervals between the reading ranges of about 0 to 4000 seconds and about 1 to 99 readings are taken with an interval between a plurality of readings.

13. The apparatus of claim 8, comprising a display of a user interface to perform one of activating or deactivating based on the setup data, wherein a message is displayed to the user via the user interface indicating that the reading is taken but not displayed.

14. The apparatus of claim 13, comprising a display for an at least one of a countdown of time between the readings and a current reading and automatically taking a subsequent reading.
